(19) United States
(12) Patent Application Publication
(10) Pub. No.: US 2026/0355538 A1
(43) Pub. Date: Nov. 12, 2026

(54) METHOD AND APPARATUS FOR PROTON THERAPY OPTIMIZATION

(71) Applicant: Siemens Healthineers International AG, Steinhausen (CH)

(72) Inventors: Sara Rosas, Helsinki (FI); Roni Hytönen, Espoo (FI)

(21) Appl. No.: 19/092,127

(22) Filed: Mar. 27, 2025

(51) Int. Cl. A61N 5/10

(52) U.S. Cl. CPC A61N 5/1031; A61N 5/1065; A61N 2005/1087

(57) ABSTRACT

A control circuit facilitates optimizing a proton therapy treatment plan for a particular patient using a proton therapy treatment apparatus having a selectively deployable range shifter. In particular, the control circuit can access an availability selection regarding usage of the selectively deployable range shifter and then optimize the proton therapy treatment plan to provide an optimized proton therapy treatment plan, wherein the optimizing includes optimizing selective use of the selectively deployable range shifter.

### BACKGROUND

The use of energy to treat medical conditions comprises a known area of prior art endeavor. For example, proton-based radiation therapy comprises an important component of many treatment plans for reducing or eliminating unwanted tumors. Proton beam radiotherapy is a form of radiation therapy that utilizes protons, which are positively charged particles, to deliver high-energy radiation to cancer cells. Unlike conventional radiation therapy that uses X-rays or photons, proton therapy can allow for more precise targeting of tumors while minimizing damage to surrounding healthy tissues. This is at least partly because protons have a unique property called the Bragg peak, which allows the protons to deposit most of their energy at a specific depth within the body.

A so-called proton therapy treatment plan often serves in the foregoing regards. A proton therapy treatment plan typically comprises specified values for each of a variety of treatment-platform parameters during administration of proton-based therapy. Such treatment plans are often automatically generated through a so-called optimization process. (As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

In intensity-modulated proton therapy treatment planning, the depth to which the particles deposit the majority of their energy is controlled by modulating the kinetic energy of the produced treatment beam. The beam is split into discrete energy layers that cover the entire target volume in the proximal-distal direction (where “proximal” refers to being closer to the beam source), so that the sum of dose delivered by all energy layers fulfils the plan prescription. In some cases, when the target lies close to the surface of the patient's body, the lowest available energy (such as 70 MeV, which reaches a depth of about 4.1 cm) can be insufficient for producing energy layers to the proximal part of the field. In these cases, clinicians sometimes use a physical apparatus called a range shifter to degrade the energy (and thus the in-patient range) of the beam to a sufficiently lower level.

### BRIEF DESCRIPTION OF DRAWINGS

The above needs are at least partially met through provision of the method and apparatus for proton therapy optimization described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings;

FIG. 3 comprises a schematic example as configured in accordance with various embodiments of these teachings; and

FIG. 4 comprises a screen shot of a graph as configured in accordance with various embodiments of these teachings.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, these various embodiments can provide for facilitating optimizing a proton therapy treatment plan for a particular patient using a proton therapy treatment apparatus having a selectively deployable range shifter. In particular, a control circuit can be configured to access an availability selection regarding usage of the selectively deployable range shifter and then optimize the proton therapy treatment plan to provide an optimized proton therapy treatment plan, wherein the optimizing includes optimizing selective use of the selectively deployable range shifter.

By one approach, the aforementioned availability selection can comprise an automated selection regarding usage of the selectively deployable range shifter. By another approach, the aforementioned availability selection can comprise a user selection regarding usage of the selectively deployable range shifter. In that case, the control circuit can be further configured to provide a user with an opportunity to select the availability selection regarding usage of the selectively deployable range shifter.

By one approach, the optimized proton therapy treatment plan can comprise an optimized intensity-modulated proton therapy treatment plan.

By one approach, the optimized proton therapy treatment plan can include treatment portions that include use of the selectively deployable range shifter and treatment portions that do not include use of the selectively deployable range shifter. If desired, the aforementioned optimizing of selective use of the selectively deployable range shifter can comprise optimizing automated selective use of the selectively deployable range shifter.

By one approach, the aforementioned optimizing of the proton therapy treatment plan can further include optimizing use of a FLASH dose rate. The latter can comprise optimizing use of a FLASH dose rate in a treatment portion that does not include use of the selectively deployable range shifter and/or not using a FLASH dose rate in a treatment portion that includes use of the selectively deployable range shifter.

By one approach, these teachings can comprise a non-transitory computer-readable medium having a computer program that itself comprises instructions that, when the computer program is executed by a computer, causes the computer to carry out any one or more of the steps, actions, and functions described herein, including, without limitation, accessing an availability selection regarding usage of the selectively deployable range shifter and then optimizing the proton therapy treatment plan to provide an optimized proton therapy treatment plan, wherein the optimizing includes optimizing selective use of the selectively deployable range shifter.

So configured, these teachings can provide for automatically determining which parts of one or more treatment fields require a range shifter to be able to fully cover the target volume and then splitting the field into one or more energy layers that are delivered with a range shifter and one or more energy layers that are delivered without a range shifter, thereby gaining the benefit of a range shifter when appropriate and avoiding the impact on plan quality from dose degradation that can be consequently caused by a range shifter when selectively choosing to not use the range shifter.

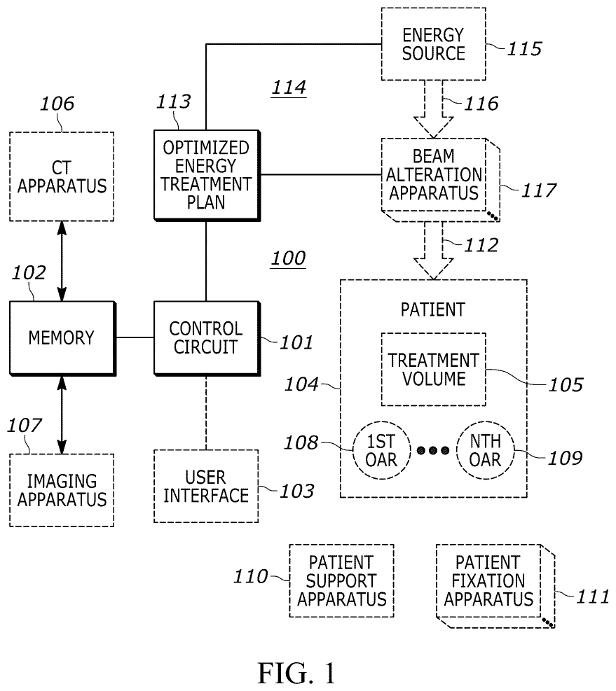

These and other benefits may become clearer upon making a thorough review and study of the following detailed description. Referring now to the drawings, and in particular to FIG. 1, an illustrative apparatus 100 that is compatible with many of these teachings will first be presented.

It may be useful, however, to first note that when a range shifter is utilized in a typical prior art plan, the range shifter is often deployed for the entirety of the treatment field, or even for all treatment fields in a proton therapy treatment plan. The applicant has determined that such usage is not without unwanted consequences. For example, range shifters can degrade the dosimetric quality of individual spots, causing individual spot doses to spread in lateral and distal-proximal directions. This phenomenon can constitute a direct negative impact on the normal tissue dose surrounding the target.

The current approach to usage may be owing to the fact that range shifters take the form of a heavy physical plate that is necessarily positioned inside the snout of the treatment delivery system. The heft of that plate and the operating position of that plate can require considerable time to deploy or remove that plate. Accordingly, even though selectively movable range shifters exist, their use in practice is limited and the prevailing practice remains to use, or not to use, a range shifter for the entirety of a treatment session.

Referring to FIG. 1, in this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

It will be appreciated that the control circuit 101 may comprise a single integrated platform or may comprise a plurality of such circuits that work in cooperation with one another.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101).

In addition to information such as radiation dosing information, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices, touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan 113. This description, for the sake of an illustrative example, will presume that the energy-based treatment plan 113 comprises a proton therapy treatment plan. This energy-based treatment plan 113 typically comprises specified values for each of a variety of treatment-platform parameters during each of a plurality of sequential exposure fields. In this case the energy-based treatment plan 113 is generated through an optimization process. Various automated optimization processes specifically configured to generate such an energy-based treatment plan are known in the art. As the present teachings are not overly sensitive to any particular selections in these regards, further elaboration in these regards is not provided here except where particularly relevant to the details of this description.

By one approach the control circuit 101 can operably couple to an energy-based treatment platform 114 that is configured to deliver therapeutic energy 112 to a corresponding patient 104 in accordance with the optimized energy-based treatment plan 113. These teachings are generally applicable for use with any of a wide variety of energy-based treatment platforms/apparatuses.

In a typical application setting the energy-based treatment platform 114 will include an energy source 115 such as a source of ionizing radiation, a source of microwave energy, a source of heat energy, and so forth. For the sake of an illustrative example, it will be presumed here that the energy source 115 is a source of protons that provides a beam of protons to irradiate diseased tissue.

By one approach this energy source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the energy source 115 along that arcuate pathway, and may accordingly control when the energy source 115 starts moving, stops moving, accelerates, de-accelerates, and/or a velocity at which the energy source 115 travels along the arcuate pathway.

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the energy source 115, and one or more energy-alteration apparatuses 117 (for example, beam-shaping apparatuses such as jaws, multi-leaf collimators, and so forth as well as range filters and range shifters) to provide selective alterations as desired. In the present description, the beam-alteration apparatus 117 will be presumed to comprise at least one range shifter. More particularly, a range shifter that can be automatically deployed and withdrawn under the control of the control circuit 101. (Range shifters are known in the art and do not require any further elaboration here.)

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

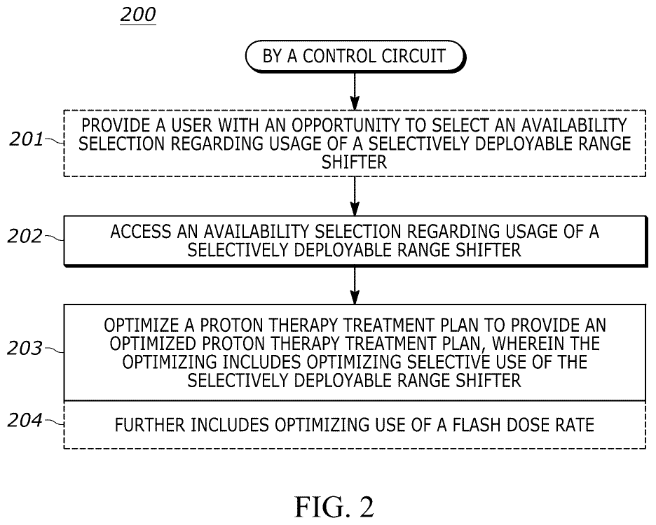

Referring now to FIG. 2, a process 200 that can be carried out, for example, in conjunction with the above-described application setting (and more particularly via the aforementioned control circuit 101) will be described. Generally speaking, this process 200 serves to facilitate optimizing a proton therapy treatment plan for a particular patient using a proton therapy treatment apparatus having a selectively deployable range shifter.

At optional block 201, this process 200 provides a user (such as an attending clinician) with an opportunity to select an availability selection regarding usage of a selectively deployable range shifter as described above. That opportunity may be provided via, for example, the above-described user interface 103. The user opportunity may take a hardware form such as one or more buttons, toggle switches, potentiometers, faders, and so forth. In lieu of the foregoing, or in combination therewith, the user opportunity may also take the form of a button, switch, checkbox, menu item, and so forth that is displayed on a display screen (such as a touch screen display).

At block 202, the control circuit 101 access an availability selection regarding usage of a selectively deployable range shifter. By one approach, the availability selection comprises an automated selection regarding usage of the selectively deployable range shifter. In lieu of the foregoing (or as a selectable option), the availability selection can comprise a user selection regarding usage of the selectively deployable range shifter (utilizing, for example, one or more selections entered by the user via the above-described selection opportunity in optional block 201).

At block 203, the control circuit 101 can then optimize a proton therapy treatment plan to provide an optimized proton therapy treatment plan, wherein the optimizing includes optimizing selective use of the selectively deployable range shifter. The latter may comprise, for example, optimizing automated selective use of the selectively deployable range shifter.

These teachings will accommodate a variety of approaches as regards the foregoing optimization. By one approach, for example, such optimization can include treatment portions that include use of the selectively deployable range shifter and treatment portions that do not include use of the selectively deployable range shifter.

As one illustrative example in these regards, the foregoing optimization can yield an optimized intensity-modulated proton therapy treatment plan. An intensity-modulated proton therapy treatment plan is an approach where the depth and intensity of the proton beam are carefully manipulated to conform to the three-dimensional shape of the tumor. Such plans modulate the energy and spatial distribution of multiple proton beams, allowing for highly precise targeting that maximizes the dose to the tumor while minimizing exposure to surrounding healthy tissues and critical structures. This level of control is often achieved through the use of a multi-leaf collimator and/or through variation of the beam's intensity by adjusting the speed or number of protons delivered.

Optional block 204 illustrates that the optimizing can further include optimizing the use of a FLASH dose rate. A FLASH dose rate refers to an ultra-high dose rate of proton delivery, significantly faster than conventional dose rates, typically exceeding 40 Gy/s. The application of radiation at such an accelerated rate—orders of magnitude higher than standard practice—is theorized to provide a differential biological effect that spares healthy tissues while still effectively targeting tumor cells. The FLASH effect suggests a potential for reducing normal tissue toxicity and increasing the therapeutic window. When integrated into an intensity-modulated proton therapy treatment plan, the FLASH dose rate aims to harness these benefits to enhance the precision and safety of proton therapy.

By one approach, the aforementioned use of a FLASH dose rate can comprise optimizing use of a FLASH dose rate in a treatment portion that does not include use of the selectively deployable range shifter. By another approach, in combination with the foregoing or otherwise, the aforementioned use of a FLASH dose rate can comprise not using a FLASH dose rate in a treatment portion that includes use of the selectively deployable range shifter.

Further details that comport with these teachings will now be presented. It will be understood that the specific details of these examples are intended to serve an illustrative purpose and are not intended to suggest any particular limitations with respect to these teachings.

By one approach, these teachings can comprise an intensity-modulated proton therapy treatment planning system that automatically sets the use of range shifters for a subset of energy layers in a field based on where the range modulation is necessary. For example, when the treatment machine energy range is sufficient to cover only the distal half of the target volume, the energy layers covering that half can be planned without a range shifter, and then the range shifter can be added for use so that the proximal half can be covered as well.

By one approach, these teachings will accommodate employing a sliding overlap region between the original and range-shifted energy layers to account for inhomogeneities along the beam path. For example, the energy layers without a range shifter can extend to a depth range of 10-6 cm, and the layers with a range shifter can extend proximally from the depth of 7 cm, resulting in an overlap region of 1 cm. This overlap region can help to smooth out movement artifacts in the dose distribution when the patient moves slightly while the range shifter is being deployed.

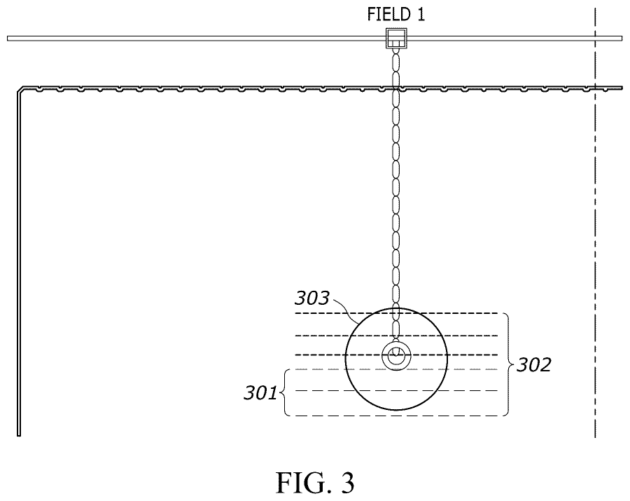

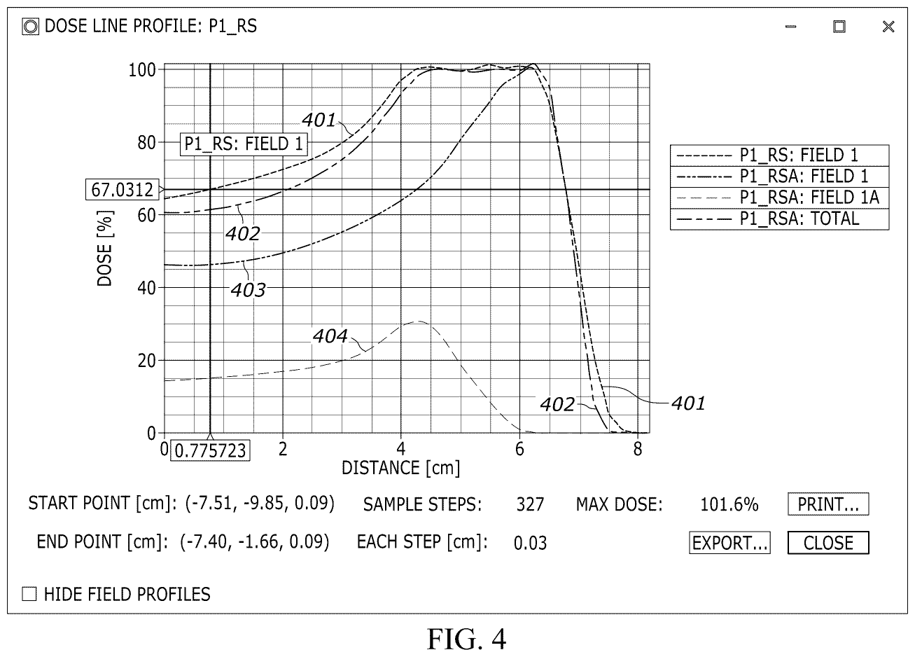

FIGS. 3 and 4 present an illustrative example where the plan target is so close to the surface of the body that the energy layers 301 of Field 1 without a range shifter can not fully cover the target volume 303. In conventional planning, this would prompt the planner to add a range shifter to obtain energy layers 302 that will cover the entire volume 303. That said, the layers with a range shifter tend to produce increased dose especially in normal tissue that is located proximally and distally from the target volume 303.

The proposed approach is compared to the conventional approach in FIG. 4. A dashed line (P1_RS) 401 represents the dose distribution along the beam path for the conventional plan, when all energy layers have the range shifter added to them in accordance with conventional practice. A solid line (P1_RSA) 402 represents the dose distribution of a plan created in accordance with the present teachings. In particular, this figure presents a dosimetric comparison of a plan when planned with a range shifter on all energy layers (P1_RS) and with the suggested adaptive range shifter approach of the present teachings (P1_RSA). Per this example, P1_RSA is represented with two fields: Field 1 contains the energy layers without a range shifter, and Field 1A contains the layers with a range shifter. Field 1 contributes most of its dose to the distal edge of the target, while Field 1A contributes dose to the proximal edge that the other field cannot reach.

Both the proximal and distal normal tissue doses are preferrable when use of the range shifter is selective and intermittent in accordance with the present teachings. Dose contributions from layers with and without the range shifter in the proposed solution are represented by the two solid lines denoted by reference numerals 403 and 404, respectively.

Further aspects of these teachings are provided by the subject matter of the following clauses (where it will be understood that any of these clauses can be combined with any one of more of the other clauses as desired).
Clause 1. A method to facilitate optimizing a proton therapy treatment plan for a particular patient using a proton therapy treatment apparatus having a selectively deployable range shifter, the method comprising: by a control circuit: accessing an availability selection regarding usage of the selectively deployable range shifter; optimizing the proton therapy treatment plan to provide an optimized proton therapy treatment plan, wherein the optimizing includes optimizing selective use of the selectively deployable range shifter.
Clause 2. The method of clause 1 wherein the optimizing of selective use of the selectively deployable range shifter comprises optimizing automated selective use of the selectively deployable range shifter.
Clause 3. The method of clause 1 wherein the availability selection comprises a user selection regarding usage of the selectively deployable range shifter.
Clause 4. The method of clause 3 further comprising: providing a user with an opportunity to select the availability selection regarding usage of the selectively deployable range shifter.
Clause 5. The method of clause 1 wherein the availability selection comprises an automated selection regarding usage of the selectively deployable range shifter.
Clause 6. The method of clause 1 wherein the optimized proton therapy treatment plan includes treatment portions that include use of the selectively deployable range shifter and treatment portions that do not include use of the selectively deployable range shifter.
Clause 7. The method of clause 1 wherein the optimized proton therapy treatment plan comprises an optimized intensity-modulated proton therapy treatment plan.
Clause 8. The method of clause 1 wherein optimizing the proton therapy treatment plan further includes optimizing use of a FLASH dose rate.
Clause 9. The method of clause 8 wherein optimizing use of a FLASH dose rate comprises optimizing use of a FLASH dose rate in a treatment portion that does not include use of the selectively deployable range shifter.
Clause 10. The method of clause 8 wherein optimizing use of a FLASH dose rate comprises not using a FLASH dose rate in a treatment portion that includes use of the selectively deployable range shifter.
Clause 11. An apparatus to facilitate optimizing a proton therapy treatment plan for a particular patient using a proton therapy treatment apparatus having a selectively deployable range shifter, the apparatus comprising: a control circuit configured to: access an availability selection regarding usage of the selectively deployable range shifter; optimize the proton therapy treatment plan to provide an optimized proton therapy treatment plan, wherein the optimizing includes optimizing selective use of the selectively deployable range shifter.
Clause 12. The apparatus of clause 11 wherein the control circuit is configured to optimize selective use of the selectively deployable range shifter by optimizing automated selective use of the selectively deployable range shifter.
Clause 13. The apparatus of clause 11 wherein the availability selection comprises a user selection regarding usage of the selectively deployable range shifter.
Clause 14. The apparatus of clause 13 wherein the control circuit is further configured to: provide a user with an opportunity to select the availability selection regarding usage of the selectively deployable range shifter.
Clause 15. The apparatus of clause 11 wherein the availability selection comprises an automated selection regarding usage of the selectively deployable range shifter.
Clause 16. The apparatus of clause 11 wherein the optimized proton therapy treatment plan includes treatment portions that include use of the selectively deployable range shifter and treatment portions that do not include use of the selectively deployable range shifter.
Clause 17. The apparatus of clause 11 wherein the optimized proton therapy treatment plan comprises an optimized intensity-modulated proton therapy treatment plan.
Clause 18. The apparatus of clause 11 wherein optimizing the proton therapy treatment plan further includes optimizing use of a FLASH dose rate.
Clause 19. The apparatus of clause 18 wherein optimizing use of a FLASH dose rate comprises optimizing use of a FLASH dose rate in a treatment portion that does not include use of the selectively deployable range shifter.
Clause 20. The apparatus of clause 18 wherein optimizing use of a FLASH dose rate comprises not using a FLASH dose rate in a treatment portion that includes use of the selectively deployable range shifter.

Those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

1. A method to facilitate optimizing a proton therapy treatment plan for a particular patient using a proton therapy treatment apparatus having a selectively deployable range shifter, the method comprising:
by a control circuit:
accessing an availability selection regarding usage of the selectively deployable range shifter;
optimizing the proton therapy treatment plan to provide an optimized proton therapy treatment plan, wherein the optimizing includes optimizing selective use of the selectively deployable range shifter.

2. The method of claim 1 wherein the optimizing of selective use of the selectively deployable range shifter comprises optimizing automated selective use of the selectively deployable range shifter.

3. The method of claim 1 wherein the availability selection comprises a user selection regarding usage of the selectively deployable range shifter.

4. The method of claim 3 further comprising:
providing a user with an opportunity to select the availability selection regarding usage of the selectively deployable range shifter.

5. The method of claim 1 wherein the availability selection comprises an automated selection regarding usage of the selectively deployable range shifter.

6. The method of claim 1 wherein the optimized proton therapy treatment plan includes treatment portions that include use of the selectively deployable range shifter and treatment portions that do not include use of the selectively deployable range shifter.

7. The method of claim 1 wherein the optimized proton therapy treatment plan comprises an optimized intensity-modulated proton therapy treatment plan.

8. The method of claim 1 wherein optimizing the proton therapy treatment plan further includes optimizing use of a FLASH dose rate.

9. The method of claim 8 wherein optimizing use of a FLASH dose rate comprises optimizing use of a FLASH dose rate in a treatment portion that does not include use of the selectively deployable range shifter.

10. The method of claim 8 wherein optimizing use of a FLASH dose rate comprises not using a FLASH dose rate in a treatment portion that includes use of the selectively deployable range shifter.

11. An apparatus to facilitate optimizing a proton therapy treatment plan for a particular patient using a proton therapy treatment apparatus having a selectively deployable range shifter, the apparatus comprising:
a control circuit configured to:
access an availability selection regarding usage of the selectively deployable range shifter;
optimize the proton therapy treatment plan to provide an optimized proton therapy treatment plan, wherein the optimizing includes optimizing selective use of the selectively deployable range shifter.

12. The apparatus of claim 11 wherein the control circuit is configured to optimize selective use of the selectively deployable range shifter by optimizing automated selective use of the selectively deployable range shifter.

13. The apparatus of claim 11 wherein the availability selection comprises a user selection regarding usage of the selectively deployable range shifter.

14. The apparatus of claim 13 wherein the control circuit is further configured to:
provide a user with an opportunity to select the availability selection regarding usage of the selectively deployable range shifter.

15. The apparatus of claim 11 wherein the availability selection comprises an automated selection regarding usage of the selectively deployable range shifter.

16. The apparatus of claim 11 wherein the optimized proton therapy treatment plan includes treatment portions that include use of the selectively deployable range shifter and treatment portions that do not include use of the selectively deployable range shifter.

17. The apparatus of claim 11 wherein the optimized proton therapy treatment plan comprises an optimized intensity-modulated proton therapy treatment plan.

18. The apparatus of claim 11 wherein optimizing the proton therapy treatment plan further includes optimizing use of a FLASH dose rate.

19. The apparatus of claim 18 wherein optimizing use of a FLASH dose rate comprises optimizing use of a FLASH dose rate in a treatment portion that does not include use of the selectively deployable range shifter.

20. The apparatus of claim 18 wherein optimizing use of a FLASH dose rate comprises not using a FLASH dose rate in a treatment portion that includes use of the selectively deployable range shifter.
